(19) United States
(12) Patent Application Publication
(10) Pub. No.: US 2020/0294228 A1
(43) Pub. Date: Sep. 17, 2020

(54) Non-Contact Multispectral Imaging for Blood Oxygen Level and Perfusion Measurement and Related Systems and Computer Program Products

(71) Applicant: East Carolina University, Greenville, NC (US)

(72) Inventors: Xin Hua Hu, Greenville, NC (US); Cheng Chen, Greenville, NC (US)

(21) Appl. No.: 16/816,714

(22) Filed: Mar. 12, 2020

Related U.S. Application Data
(60) Provisional application No. 62/817,685, filed on Mar. 13, 2019.

(51) Int. Cl. G06T 7/00; A61B 5/026; A61B 5/1455; A61B 5/00

(52) U.S. Cl. CPC G06T 7/0012; A61B 5/0261; A61B 5/14551; A61B 5/0077; A61B 2560/0431; G06T 2207/30104; G06T 2207/20221; G06T 2207/10048; G06T 2207/10036

(57) ABSTRACT

Systems for non-contact imaging measurement of blood oxygen saturation and perfusion in a sample are provided including a control unit configured to facilitate acquisition of data from a sample; a data acquisition module coupled to the control unit, the data acquisition module configured to illuminate a field of view (FOV) of the sample using a plurality of wavelengths to provide a plurality of images corresponding to each of the plurality of wavelengths responsive to control signals from the control unit; and an image processing module configured calculate image saturation parameters and reflectance for each of the plurality of images having a unique acquisition time and unique wavelength and extracting blood volume and oxygen saturation data in the FOV using the calculated image saturation parameters and reflectance for each of the plurality of images having a unique acquisition time and unique wavelength.

### CLAIM OF PRIORITY

The present application claims priority to U.S. Provisional application Ser. No. 62/817,685, filed Mar. 13, 2019, entitled Non-Contact Multispectral Imaging for Blood Oxygen Level and Perfusion Measurement and Related Systems and Computer Program Products, the contents of which are hereby incorporated herein by reference as if set forth in its entirety.

### FIELD

The present inventive concept relates generally to imaging and, more particularly, to multispectral imaging.

### BACKGROUND

Blood perfusion in tissue beds supplies oxygen through the capillary network for maintaining essential metabolism. Thus, quantification of perfusion can provide critical physiological information in assessment of conditions in people of poor health and rate of recovery in patients undergoing treatments. Pulse oximetry devices, for example, for point-based measurement of oxygen level, are used ubiquitously in operation rooms and critical care setting. Pulse oximetry devices generally measure oxygen saturation of arterial blood in a subject by utilizing, for example, a sensor attached typically to a finger, toe, or ear to determine the percentage of oxyhemoglobin in blood pulsating through a network of capillaries. Accurate mapping of blood perfusion related parameters and oxygen level by optical imaging remains very challenging because, for example, of the high turbidity (thickness/cloudiness) and heterogeneity of skin and other tissue.

### SUMMARY

Some embodiments of the present inventive concept provide systems for non-contact imaging measurement of blood oxygen saturation and perfusion in a sample, the system including a control unit configured to facilitate acquisition of data from a sample; a data acquisition module coupled to the control unit, the data acquisition module configured to acquisition module coupled to the control unit, the data acquisition module configured to illuminate a field of view (FOV) of the sample using a plurality of wavelengths to provide a plurality of images corresponding to each of the plurality of wavelengths responsive to control signals from the control unit; and an image processing module configured calculate image saturation parameters and reflectance for each of the plurality of images having a unique acquisition time and unique wavelength and extracting blood volume and oxygen saturation data in the FOV using the calculated image saturation parameters and reflectance for each of the plurality of images having a unique acquisition time and unique wavelength.

In further embodiments, the data acquisition module may further include a plurality of sets of light emitting diodes (LEDs) each having an associated wavelength; and a camera coupled to the plurality of sets of LEDs, wherein each set of LEDs is configured to illuminate the FOV of the sample at the associated wavelength responsive to a unique driving current from the control unit to provide an image of the FOV of the sample at the associated wavelength.

In still further embodiments, each of the plurality of images may be acquired at the associated plurality of wavelengths using a narrow bandwidth in a range from about 0.2 nm to about 50 nm.

In some embodiments, the camera may be a charge coupled device (CCD) camera and each of LEDs may have an optical power of at least 500 mW per wavelength.

In further embodiments, extracting blood volume and oxygen saturation data may include extracting heart-rate based mapping of blood vessel volume changes and detecting blood oxygen saturation level.

In still further embodiments, the system may be further configured to obtain a fused image of blood perfusion and oxygen saturation in skin tissues in a visible region and probe deeper tissue layers of lower dermis and cutaneous fat layers in near-infrared (NIR) regions using the plurality of images obtained at the corresponding plurality of wavelengths.

In some embodiments, the system may be handheld.

In further embodiments, the system may be configured to self-calibrate.

Related methods and systems are also provided.

### BRIEF DESCRIPTION OF THE DRAWINGS

FIG. 1 is a diagram illustrating a schematic of a front panel of a system having a multispectral illumination unit (multispectral light emitting diodes (LEDs)) on two rings centered around a charge coupled device (CCD) camera in accordance with some embodiments of the present inventive concept.

FIG. 2 is a table illustrating optical specifications in accordance with some embodiments of the present inventive concept.

FIG. 3A is a diagram illustrating a side view (cross section) of a diffused reflection due to scattering in a layered tissue bed in accordance with some embodiments of the present inventive concept.

FIG. 3B is a diagram illustrating a configuration of illumination (only one LED beam is shown) and imaging in accordance with some embodiments of the present inventive concept.

FIG. 4 is a flowchart illustrating operations of a system in accordance with some embodiments of the present inventive concept.

FIGS. 5A through 5F are images obtained from a reflection image Pₘ of a hand using systems in accordance with embodiments of the present inventive concept; FIGS. 5A through 5C are bright-field images acquired at different wavelengths λ as indicated on the images and FIGS. 5D through 5F are corresponding heart-rate reference (HRR) images, respectively, in accordance with some embodiments of the present inventive concept.

FIGS. 6A through 6C are frequency plots of time-sequence data of mean pixel values of three regions as marked (a, b, c) on FIG. 5F in accordance with some embodiments of the present inventive concept.

FIG. 7 is a block diagram illustrating a basic data processing system that may be used in accordance with some embodiments of the present inventive concept.

### DETAILED DESCRIPTION

The present inventive concept will be described more fully hereinafter with reference to the accompanying figures, in which embodiments of the inventive concept are shown. This inventive concept may, however, be embodied in many alternate forms and should not be construed as limited to the embodiments set forth herein.

Accordingly, while the inventive concept is susceptible to various modifications and alternative forms, specific embodiments thereof are shown by way of example in the drawings and will herein be described in detail. It should be understood, however, that there is no intent to limit the inventive concept to the particular forms disclosed, but on the contrary, the inventive concept is to cover all modifications, equivalents, and alternatives falling within the spirit and scope of the inventive concept as defined by the claims. Like numbers refer to like elements throughout the description of the figures.

The terminology used herein is for the purpose of describing particular embodiments only and is not intended to be limiting of the inventive concept. As used herein, the singular forms “a”, “an” and “the” are intended to include the plural forms as well, unless the context clearly indicates otherwise. It will be further understood that the terms “comprises”, “comprising,” “includes” and/or “including” when used in this specification, specify the presence of stated features, integers, steps, operations, elements, and/or components, but do not preclude the presence or addition of one or more other features, integers, steps, operations, elements, components, and/or groups thereof. Moreover, when an element is referred to as being “responsive” or “connected” to another element, it can be directly responsive or connected to the other element, or intervening elements may be present. In contrast, when an element is referred to as being “directly responsive” or “directly connected” to another element, there are no intervening elements present. As used herein the term “and/or” includes any and all combinations of one or more of the associated listed items and may be abbreviated as “/”.

Unless otherwise defined, all terms (including technical and scientific terms) used herein have the same meaning as commonly understood by one of ordinary skill in the art to which this inventive concept belongs. It will be further understood that terms used herein should be interpreted as having a meaning that is consistent with their meaning in the context of this specification and the relevant art and will not be interpreted in an idealized or overly formal sense unless expressly so defined herein.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element without departing from the teachings of the disclosure. Although some of the diagrams include arrows on communication paths to show a primary direction of communication, it is to be understood that communication may occur in the opposite direction to the depicted arrows.

Although some embodiments of the present inventive concept are discussed with respect to measurement of blood oxygen saturation in the tissue bed, embodiments of the present inventive concept are not specifically limited there. Other samples may be used without departing from the scope of the present inventive concept.

As discussed above, optical imaging device for quantitative assessment of oxygen saturation distributions and blood perfusion in a tissue bed are unavailable despite intense research efforts. Accordingly, some embodiments of the present inventive concept provide a system for non-contact imaging measurement of blood oxygen saturation and perfusion in a tissue bed. Embodiments of the present inventive concept combine multispectral imaging for determination of blood oxygen level with time-sequenced imaging for extraction of heart beat induced blood volume change distributions to quantify blood perfusion. Embodiments of the present inventive concept provide the following advantages over existing blood oximetry devices: (1) self-calibration of spectral images for extraction of intrinsic blood volume change and perfusion signals; (2) time-sequenced imaging for retrieving a heart-rate induced blood volume change map in tissue bed; (3) multispectral imaging for mapping of blood oxygen level distribution; (4) effective algorithms for mapping blood perfusion and oxygen saturation as will be discussed further below.

Blood perfusion can be measured as a point based velocity measurement by ultrasound and electromagnetic flow meter or imaging measurement by optical, computed tomography (CT), magnetic resonance imaging (MRI) and positron-emission tomography (PET), which has a market size expected to reach $12.03 billion by the end of 2023 with a compound annual growth rate (CAGR) of 8.2% from 2017 to 2023. No optical imaging product, however, has found its way into commercial use for mapping both perfusion and blood oxygen saturation because of strong turbid and heterogeneous nature of blood capillary network embedded in soft tissues. Some embodiments of the present inventive concept provide a system to demonstrate the feasibility of hand-held devices, which can acquire multispectral and time-sequenced image data and rapidly extract blood oxygen saturation and perfusion distribution as a fused image of the tissue bed.

Pulse oximetry devices operate on the principle of photoplethysmography (PPG) at the two wavelengths of red (˜660 nm in wavelength) and infrared (˜940 nm) for measurement of blood oxygen saturation. Due to accuracy and robustness, it has wide clinical applications including patient monitoring in clinics and sleep quality assessment at home. Moving from point-based measurement to non-contact PPG imaging has attracted strong research interests that can map blood vessel volume change in tissue bed. The current PPG imaging technology, however, provide only qualitative information of blood vessel volume change in the tissue bed with no information on perfusion and oxygen saturation. Multispectral imaging HyperView™, (HyperMed Imaging, Inc. Memphis, Tenn. 3812) is a handheld, battery operated, portable diagnostic imaging device that is used to assess tissue oxygenation without contacting the patient.

Furthermore, a multispectral reflectance imaging system that can inversely determine the absorption and scattering properties of skin tissues for non-invasive diagnosis of cutaneous melanoma has been developed by East Carolina University (ECU). See, e.g., U.S. Pat. No. 8,634,077, the contents of which are hereby incorporated by reference as if recited in full herein. By combining reflectance imaging with spectral scans in the visible and near-infrared regions, the spatial distribution of the tissue components of interest, such as red blood cells moving in the capillary vessels of blood in the skin dermis layer can be determined as a three dimensional (3D) data cube of two dimensions (2D) in real space and one dimension (1D) in light wavelength. Reflectance imaging research has been extended from cancer diagnosis to heart rate-based blood volume change mapping by adding a time-domain measurement of multispectral image data. Data indicates that blood volume change due to a heartbeat can be imaged at multiple wavelengths for quantitative assessment of perfusion and oxygen saturation by adapting tissue optics modeling with Fourier transforms. Using these concepts, embodiments of the present inventive concept may provide the capability to perform quantitative and non-contact determination of blood perfusion and oxygen saturation distribution. Furthermore, some embodiments use a compact light source of, for example, light emitting diodes (LEDs), and acquire rapidly the four-dimensional (4D) image cubes of “big data” nature, which enables hand-held devices and machine learning algorithms to extract additional information, such as blood pressure and cardiac stress signals using the same device platform.

As used herein, a “tissue bed” refers to layers of tissue that light can penetrate up to at least several millimeters; “turbid” refers to media that light scattering dominates light-medium interaction; “big data” refers to the large sizes of acquired data files per imaged site, for example, 500 MB or larger; and “rapidly” refers to acquiring data in less than about 5 minutes. Further, embodiments of the present inventive concept may be used to image any sample that lends itself to the inventive concept without departing from the scope of the present inventive concept.

It will be understood that although embodiments of the present inventive concept discuss the use of LEDs as one example of a “non-coherent” light source, embodiments of the present inventive concept are not limited to this configuration. Other types of light sources, such as coherent or non-coherent light sources, may be used without departing from the scope of the present inventive concept. As used herein, the term “non-coherent” refers to spatial coherent length shorter than 1.0 millimeter in visible spectral region; and the term “coherent” refers to spatial coherent length longer than 10 millimeters in visible spectral region.

Some embodiments of the present inventive concept provide an imaging system for performing multispectral and time-sequenced acquisition of images, for example, hand images, at wavelengths in a particular range, for example, from 520 nm to 940 nm, using a compact light source of, for example, LEDs. Different imaging parameters with wavelengths from 300 nm to 3000 nm and human or animal tissue types can be enabled by controlling of illumination and imaging polarization and exposure times.

Embodiments of the present inventive concept provide processors that perform image data processing algorithms to extract heart-rate based mapping of blood vessel volume changes and detect blood oxygen saturation level and changes. Furthermore, some embodiments provide self-calibration to obtain tissue reflectance from reflected light for the multispectral images by illumination intensity modulation without performing separate calibration with a reflectance standard. Accordingly, systems in accordance with embodiments of the present inventive concept may be used to obtain the fused image of blood perfusion and oxygen saturation in skin tissues in the visible region and probe deeper tissue layers of lower dermis and cutaneous fat layers in the near-infrared (NIR) regions. Although embodiments of the present inventive concept are discussed with respect to “hand” images, embodiments of the present inventive concept are not limited thereto. Embodiments may be used to image any portion of the subject without departing from the scope of the present inventive concept.

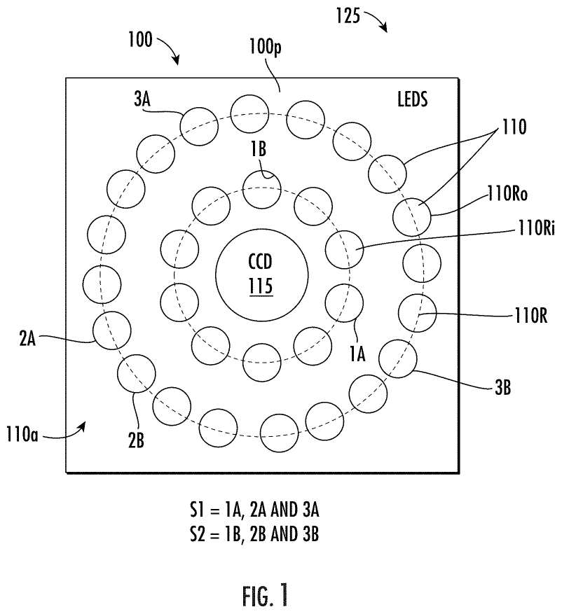

Referring now to FIG. 1, a system in accordance with some embodiments of the present inventive concept will be discussed. As illustrated in FIG. 1, the system includes a multispectral illumination unit 125 including two rings A and B centered around a charge coupled device (CCD) camera 115. The multispectral illumination unit 125 may be, for example, a multispectral LED based illumination unit, that can be synchronized with a camera exposure control for four dimensional (4D) image acquisition with two dimensional (2D) referring to the image dimensions plus one dimension (1D) to the time sequenced imaging and 1D to the multispectral imaging. The system may further include a processor that is configured to run control, data acquisition and tissue optics based image processing modules to perform robust and rapid reflectance self-calibration to remove the effect of incident light intensity on the acquired image pixel values without the need to acquire another set of images from, for example, a diffused reflectance standard of calibrated reflectance at the time of tissue imaging, Fourier transform, heart rate frequency extraction, selection of tissue regions of high blood volume change amplitude, spectral tissue absorption analysis and image fusing. The system may be optimized to, for example, automate image acquisition and subsequent extraction of blood perfusion and oxygen saturation maps.

In particular, the system in accordance with embodiments discussed herein may be used for acquisition of multispectral and time-sequenced images from skin tissues with synchronized illumination. As discussed above, the system includes at least one multispectral illumination unit 125. In some embodiments, the illumination units comprises one or more multispectral LEDs for imaging at plurality of different wavelength bands, such as about 3-30, more typically 4-15, optionally 10, wavelength bands, with center wavelengths ranging from 400 nm to 1100 nm and bandwidths of 60 nm or less, typically smaller than 60 nm, such as bandwidths in a range of 1 nm-50 nm or 10 nm-40 nm.

The multispectral illumination unit 125 may further include an optical setup for beam-shaping LED outputs with micro-lenses with high coupling efficiency. The multispectral imaging unit is equipped with a camera, for example, a 12-bit monochromatic charge coupled device (CCD) camera 115, connected to a host computer or embedded microprocessor with, for example, a universal serial bus, (USB) 3.0 cable for acquiring images of 640×480 pixels at a rate up to 120 frames per second. The exposure time of the CCD camera 115 can be adjusted, for example, from 1.0 millisecond to 10 seconds. The control unit provides LED currents that can be modulated by the data acquisition and control modules to power selected LEDs with electric currents at selected modulation frequency, duty factor and synchronized with the exposure time of camera.

As discussed above, some embodiments of the present invention include modules configured to allow (1) modulation of LED current for acquiring paired images at high and low illumination intensity at a selected wavelength; (2) synchronization of LED illumination with CCD camera exposure to scan over a plurality of different, defined wavelength bands, such as 10 wavelength bands, for multispectral image acquisition; (3) performing self-calibration of multispectral images; and (4) displaying and recording parameters of system control and image acquisition to ensure data quality. It will be understood that items (1) through (4) are provided as examples only and, therefore, do not limit embodiments of the present inventive concept.

Embodiments of the present inventive concept also include methods, systems and computer program products processing the obtained images. For example, the image processing module may perform the following: (1) a Fourier transform to extract heart rate map and blood volume change map from time-sequenced images; (2) determine blood related tissue absorption maps at different wavelengths; (3) determine blood oxygen saturation distribution in tissue bed from wavelength dependence of tissue absorption and blood volume change maps; (4) determine blood perfusion distribution and quantitative biomarkers; and (5) fuse the blood oxygen saturation and perfusion maps into a common coordinate map (CCM).

Example embodiments of the present inventive concept will now be discussed with respect to FIGS. 1 through 7 below. As discussed above, some embodiments of the present inventive concept provide a system that enables time-sequenced acquisition of polarized multispectral images from skin or other tissue types in vivo. The system may include an illumination module, an imaging module and a control module. It will be understood that these three modules may be combined into less than three modules or separated into more than three modules without departing from the scope of the present inventive concept.

Referring again to FIG. 1, a diagram of a schematic view of a system front panel including a multispectral illumination unit 125 in accordance with some embodiments of the present inventive concept will be discussed. As illustrated in FIG. 1, the front panel 100p of the system 100 includes a plurality of concentric rings 110R of multispectral light emitting diodes (LEDs) 110 around a charge coupled device (CCD) camera 115. As shown, there is an inner ring 110Ri and an outer ring 110Ro, radially spaced apart a distance from the inner ring 110Ri. The outer ring 110Ro can have more LEDs 110 than the inner ring 110Ri. In particular, as shown, the front panel 100p illustrated in FIG. 1 combines thirty high power LEDs 110 (20 on the outer ring 110Ro and 10 on the inner ring 110Ri) into an array 110a as the light source of the illumination unit 125. The rings 110R can be arranged as two rings concentric to the CCD camera 115 of the imaging unit 125. The term “high power” with respect to LEDs 110 refers to greater than or equal to 10 milliWatts (mW), typically 100 mW-1 W. Typically, the LEDs are configured to operate using up to 2.0 amps (A) of current.

Centers of one or more LEDs 110 in the inner ring 110Ri can be aligned with adjacent centers of an LED 110 in the outer ring 110Ro. Centers of other LEDs 110 in the inner ring 110Ri can be circumferentially offset from centers of adjacent LEDs in the outer ring 110Ro.

The LEDs 110 can be provided as a plurality of sets, such as ten sets of three for thirty LEDs, of different wavelengths ranging from 400 nm to 1100 nm with bandwidths of 40 nm or less. The sets can include one or more LEDs 110 in each ring 110R. For example, in some embodiments, first and second sets S1 and S2, respectively, of LEDs may include three LEDs each, one on inner ring 110Ri and two on the outer ring 110Ro. An example first set S1 is illustrated in FIG. 1 as including LED 1A on the inner ring 110Ri and two LEDs 2A and 3A on the outer ring 110Ro. Similarly, an example of a second set S2 is also illustrated in FIG. 1 as including LED 1B on the inner ring 110Ri and two LEDs 2B and 3B on the outer ring 110Ro. The first and second sets may include LEDs having a same wavelength within the set and different wavelengths between the sets. However, embodiments of the present inventive concept are not limited to this configuration.

The LED driving currents are supplied and modulated by a control unit circuit so that only one set of LEDs of the same wavelength is illuminating the field-of-view (FOV). The currents of LEDs 110 are synchronized among each other and to camera exposure time to produce intensity modulation for self-calibration and wavelength scan for multispectral imaging. In some embodiments, the intensity modulation and scan over the plurality of different wavelength bands, i.e., ten wavelength bands, may be completed rapidly, typically within less than 5 minutes, such as about 180 seconds. Furthermore, the scan time may be further reduced when illumination wavelength bands are optimized to, for example, six or less with minimal reduction in extraction of blood related information from the acquired image data.

Each of the LEDs 110 in the array 110a may be combined with a micro lens that has a numerical aperture and focal length for high transmission and beam collimation onto the FOV. Furthermore, both LEDs 110 and CCD camera 115 may have linear polarization to enable s-polarized and p-polarized illumination and image acquisition. The use of polarization control allows effective separation of diffusely reflected light from superficial and deep tissue layers. Because of the variable depth of blood capillary network under tissue surface, acquisition of same- or cross-polarized images may enhance the ability of prototype system to map blood volume change distribution in the highly turbid tissue bed.

Although embodiments of the present inventive concept are discussed above as having thirty LEDs 110 and using specific wavelengths, it will be understood that these numbers are provided for example only and, therefore, embodiments of the present inventive concept are not limited thereto.

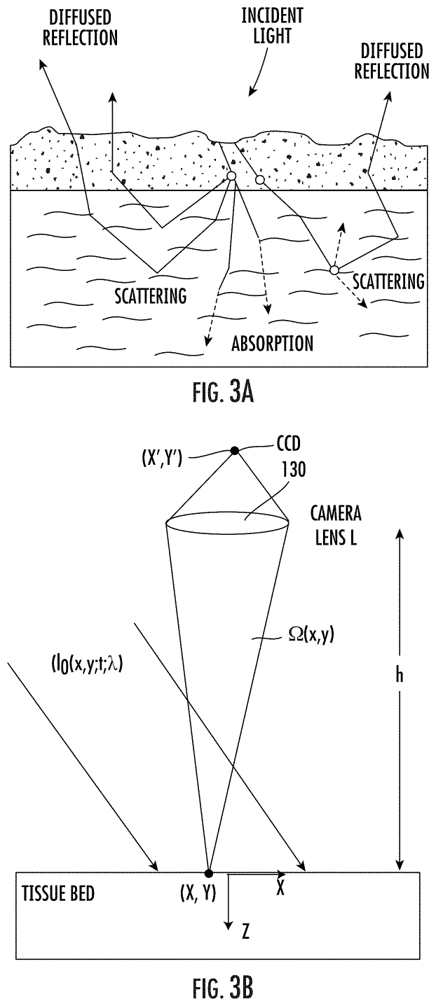

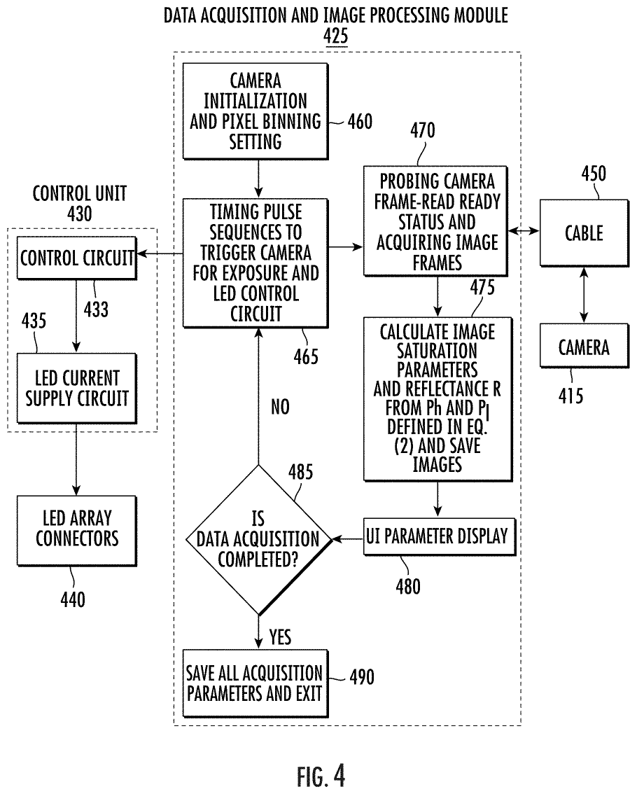

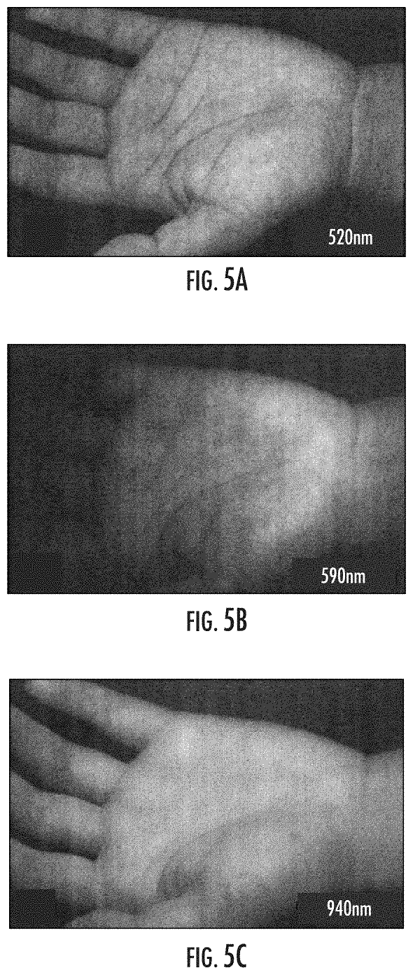

In some embodiments, the imaging unit comprises a 12-bit monochromatic CCD camera (115, FIG. 1) having high pixel sensitivity from 400 nm to 1100 nm and a camera lens 130 (FIG. 3B) of appropriate focal length and numerical aperture for rapid image acquisition at a rate of 30 frames per second or higher. The camera may be controlled by a control module 430 (FIG. 4), for example, a master clock timing signal to the control unit circuit 430 (FIG. 4) for synchronization of LED current modulation and image transfer through an output, optionally a USB 3.0 cable 450 (FIG. 4). In some embodiments, the CCD camera 115 has a pixel binning function for images of 640×480 pixels to increase a dynamic range of pixel values and frame transfer rate. The control unit 430 (FIG. 4) may include, for example, a DC current power supply circuit 435 (FIG. 4) for providing the high-power LEDs with peak current values up to 6 Amps (A) (2 A per LED) and a control circuit for modulation of the LED current by a trigger signal from a digital-to-analog (D/A) circuit the camera 115 at selected values of duty factor.

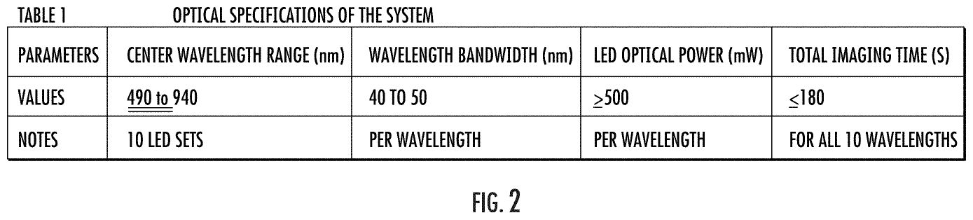

FIG. 2 includes Table 1, which provides a list of the main specifications of an example system in accordance with some embodiments of the present inventive concept. In particular, Table 1 provides a center wavelength range of from about 490 to about 940 nm with 10 LED sets; a wavelength bandwidth of about 40 to 50 nm per wavelength; LEDs having an optical power of at least 500 mW per wavelength; and a total imaging time of 180 seconds for all 10 wavelengths. It will be understood that Table 1 provides example specifications and embodiments of the present inventive concept are not limited thereto.

Nearly all human or animal soft tissues including skin and epithelial tissues with embedded blood vessels are of strong turbid nature due to elastic scattering of incident light dominating the light-tissue interaction. FIG. 3A illustrates a side view (cross section) of a diffused reflection due to scattering in a layered tissue bed of a sample and FIG. 3B illustrates a configuration of illumination (only one LED beam is shown) and imaging in accordance with some embodiments of the present inventive concept. As illustrated in FIGS. 3A and 3B, a portion of the light illuminating (incident light) the sample is scattered inside tissue and exits from the surface of illumination as “diffused reflection.” The intensity of the diffused reflected light I_{R} (x′, y′; t; λ) depends on the optical properties of tissues and on the intensity of incident light I₀ (x, y, z=0; t; λ). As used herein, (x′, y′) and (x, y) refer to the planes perpendicular to the z-axis (Vertical arrow pointed down into the sample) in FIG. 3A for camera sensor at z=h and tissue surface at z=0 respectively, t is time of image acquisition and λ is the wavelength of illumination. Prior applications have used a diffused reflectance standard to remove the effect of incident light I₀ by obtaining the diffused reflectance R of the tissue from the reflected light I_{R} by measurement of incident light I₀ using the standard of known reflectance R_{std}. While this method is very effective, the measurement of incident beam profile I₀ is time consuming. Thus, some embodiments of the present inventive concept provide a self-calibration method that allows obtaining diffused reflectance of tissue R without the need for two measurements of reflected light from tissue and reflectance standard.

Referring now to FIG. 3B, operations of this method will be discussed. As illustrated in FIG. 3B, the optical configuration of illumination and imaging for the system is plotted. In particular, for each pixel at (x′, y′) on the sensor plane of z=h, the measured light intensity I_{R} corresponds to those light or photons exiting at (x, y) from the tissue surface with the solid angle Ω(x, y) as shown in FIG. 3B to the camera lens L. Thus:

$$P ( {x}^{′} , {y}^{′} ; t ; λ ) = {P}_{m} ( {x}^{′} , {y}^{′} ; t ; λ ) - {P}_{n} ( {x}^{′} , {y}^{′} ; t ; λ ) = \frac{k ( λ ) R ( x , y ; t ; λ ) {I}_{0} ( x , y ; t ; λ ) Ω ( x , y )}{2 π} \qquad Eqn . ( 1 )$$

where P denotes the pixel value after removal of background noise Pₙ from the measure pixel value Pₘ; k(λ) denotes the spectral response function of CCD sensor to reflected light intensity I_{R}; and R(x, y; t) denotes the tissue's diffused reflectance and 27π is the solid angle of the half space from any surface location. In Eqn. (1), it is assumed that the camera sensor plane coordinates (x′, y′) and the sample surface coordinates (x, y) form a one-to-one relation due to the conjugate relation of object and image by the camera lens L after system alignment.

To determine R (x, y, z=0; t; λ) of the imaged tissue bed from the acquired image of P(x, y; t; λ) in the variable space of 4D nature, the following equation has been developed to show a relation between R and two images from the same tissue bed denoted as Pₕ for reflection image acquired with high illumination intensity and Pₗ with low illumination intensity:

$$R ( x , y ; t ; λ ) = \frac{{\{ {P}_{h} ( x , y ; t ; λ ) - {P}_{l} ( x , y ; t ; λ ) \}}_{tis}}{{\{ {P}_{h} ( x , y ; λ ) - {P}_{l} ( x , y ; λ ) \}}_{std}} {R}_{std} \qquad Eqn . ( 1 )$$

where { . . . }ₜᵢₛ is obtained from two images acquired from the tissue bed at time t and wavelength λ, and { . . . }_{std} is obtained from two images acquired from a diffused reflectance standard with calibrated reflectance R_{std}. Since the two images from reflectance standard are time independent, they only need to be acquired once for each λ value for the prototype system before tissue imaging, instead of being acquired every time after imaging a site of tissue bed. Furthermore, an LED's optical light intensity I₀ scales linearly with its input electric current i and can be accurately controlled by modulating i. Consequently, tissue reflectance R(x, y, z=0; t; λ)=R (x, y; t; λ) can be determined or self-calibrated using Eqn. (2) which may also eliminate the background noise as denoted as Pₙ in Eqn. (1).

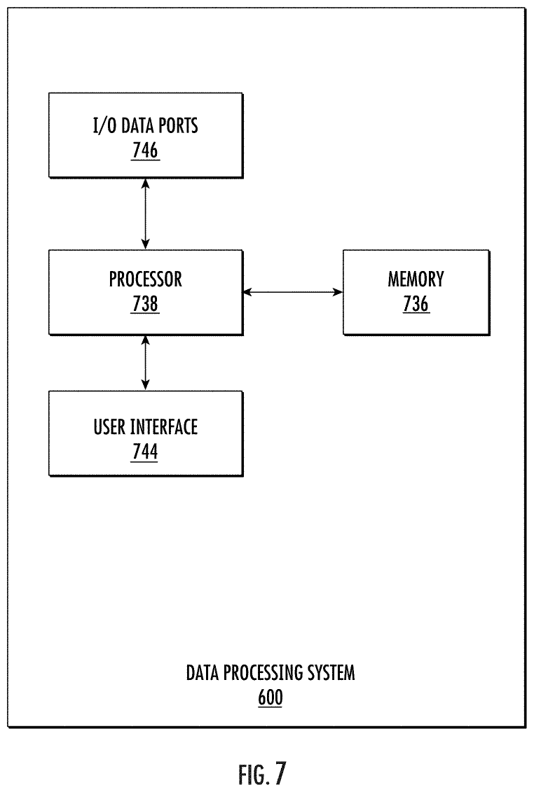

Referring now to the diagram of FIG. 4, systems and operations of the control and data acquisition modules in accordance with some embodiments of the present inventive concept will be discussed. In particular, FIG. 4 illustrates the logic flow of the control and data acquisition modules and the relationship to the devices of control unit, the connector (USB) and camera (CCD) in accordance with some embodiments of the present inventive concept. A user may control the system using, for example, a user interface (UI) 744 (FIG. 7) to start an imaging process with selected wavelengths and LED modulation parameters, such as exposure time and LED current for Pₕ and Pₗ. After image acquisition, the control module may be used to calculate diffused reflectance R(x, y; t; λ) for each acquisition time t and illumination wavelength λ which can be used by the image processing module to extract blood volume change and oxygen saturation maps in accordance with embodiments of the present inventive concept.

It will be understood that FIG. 4 illustrates some embodiments and is provided as an example and does not limit embodiments of the present inventive concept to the details therein. In detail, as illustrated in FIG. 4, the data acquisition and image processing modules 425 communicate with the control unit 430, which communicates with the LED array connectors 440. As further illustrated in FIG. 4, the data acquisition and image processing modules 425 communicate with the camera 415 (for example, a CCD camera) via a data cable 450 (for example, a USB 3.0 data cable). Operations of the data acquisition and image processing modules 425 begin at block 460 by initializing the camera and pixel binning setting. The pulse sequences are timed to trigger the camera (415) for exposure and LED control circuit (block 465). The camera (415) is probed for frame-ready status and image frames may be acquired (block 470). The image saturation parameters and reflectance R from P1 and Pn as set out above in Eqn. (2) may be calculated and the images may be saved (block 475). The parameters are displayed on a user interface (UI) (block 480). It is determined if the data acquisition is complete (block 485). If it is determined that the data acquisition is complete (block 485), operations continue to block 490 where all acquisition parameters are saved and the system is exited. If, on the other hand, it is determined that the data acquisition is not complete (block 485), operations return to block 465 and repeat until it is determined that the data acquisition is complete (block 485).

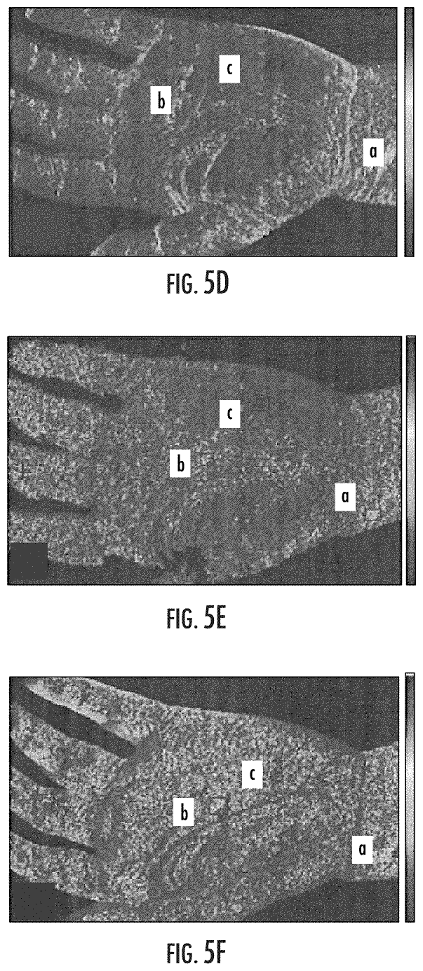

In some embodiments of the present inventive concept, an HRR image will be established to register and extract blood perfusion and oxygen saturation maps from the multispectral reflection image data of Pₘ(x′, y′; t; λ). In some embodiments, the HRR can be obtained at different wavelength of λ after filtering the time-sequenced images with a narrow band in frequency domain using the fast Fourier transform (FFT) technique. A peak frequency f₀ can be recognized from tissue regions marked as a and b in FIGS. 5D to 5F. Most of the tissue bed regions in the hand images do not contain such peaks, marked as regions c. It is clear from these results that the regions a and b have high density of blood capillary network and f₀ is the heartbeat rate of the sample being imaged. It is also clear that the blood volume change due to the heart beat shows a larger number of pixels having higher amplitudes at f₀ at the near-infrared region of 940 nm (FIGS. 5C and 5F) in comparison to the visible regions of 520 nm (FIGS. 5A and 5D) and 590 nm (FIGS. 5B and 5E). The difference is directly related to the deeper penetration of near-infrared light of skin tissues, which provide a higher average number of pixels that correlates with the blood volume changes.

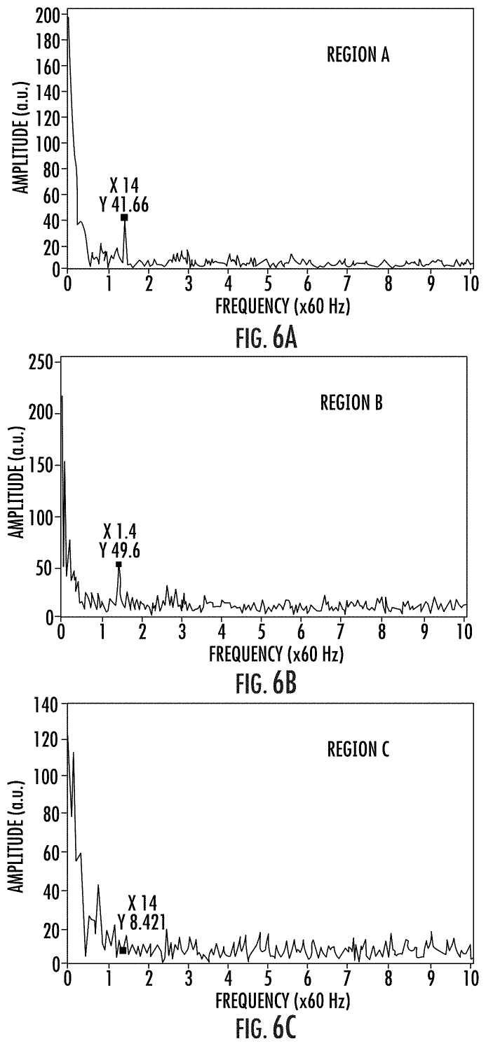

Referring now to FIGS. 6A through 6C, graphs of amplitude versus frequency will be discussed. These figures illustrated the frequency (x60Hz) plots of time sequence data of mean pixel values of three the three regions a, b and c in FIG. 6F (λ=940 nm).

Some embodiments of the present inventive concept may further improve the HRR image contrast using the self-calibration method to replace Pₘ(x′, y′; t; λ) by diffused reflectance R(x, y; t; λ). Some embodiments also enhance the FFT based algorithm's robustness for searching heart-rate frequency f₀ of all pixels in the FOV with a cascade bandwidth scheme. With the HRR images established at each wavelength of illumination, co-registration of blood volume change may be performed to generate a common coordinate map (CCM) for all multispectral HRR images that will be used to obtain blood oxygen saturation map by applying the radiative transfer model of light scattering.

Due to the strong turbid nature of human tissue, a widely used light scattering model of radiative transfer theory can be used to characterize the light-tissue interaction:

$$s \cdot ∇ L ( r , s ) = - ( {μ}_{a} + {μ}_{s} ) L ( r , s ) + {μ}_{s} {\int }_{4 π} p ( s , {s}^{′} ) L ( r , {s}^{′} ) d {ω}^{′} , \qquad Eqn . ( 2 )$$

where μₐ, μₛ and pare, respectively, the absorption, scattering and scattering phase function of the imaged tissue and L(r, s) is light radiance at location r along direction given by the unit vector s. Over the past decades, Monte Carlo based tissue optics software has been developed that allows extraction of μₐ, μₛ and p from the measured light signals L in terms of Pₘ discussed in Eqn. (1) at different wavelengths λ. Some embodiments of the present inventive concept are configured to extract a tissue absorption parameter map B(x, y; λ) based on the multispectral HRR image data that is related to the blood component of μₛ(λ). By combining B(x, y; λ) and CCM the distribution of blood oxygen saturation in the imaged tissue bed may be obtained.

Referring now to FIG. 7, an example embodiment of a data processing system 700 suitable for use in accordance with some embodiments of the present inventive concept will be discussed. For example, the data processing system 700 may be provided anywhere in the system without departing from the scope of the present inventive concept. As illustrated in FIG. 7, the data processing system 700 includes a user interface 744 such as a display, a keyboard, keypad, touchpad or the like, I/O data ports 746 and a memory 736 that communicates with a processor 738. The I/O data ports 746 can be used to transfer information between the data processing system 700 and another computer system or a network. These components may be conventional components, such as those used in many conventional data processing systems, which may be configured to operate as described herein. This data processing system 700 may be included in any type of computing device without departing from the scope of the present inventive concept.

As briefly discussed above, embodiments of the present inventive concept provide methods, systems and computer program products for image capture and processing that integrate illumination and imaging synchronization, time-sequenced and multispectral image acquisition and analysis to aid extraction of blood perfusion and oxygen saturation maps. Systems in accordance with embodiments discussed are non-contact in nature; provide novel methods of calibrating raw images into reflectance images without use of reflectance standard; add time-domain image measurements to determine heart-beat distribution in samples (tissues); apply multispectral imaging with LED light source; provide 3D to 4D image measurement; use the heart-beat as a modulation to demodulate multispectral images for blood perfusion imaging apply spectral analysis for blood oxygen imaging; and provide a radiative transfer model based analysis of blood perfusion and oxygenation. Embodiments of the present inventive concept may be extended to disease diagnosis in addition to physiology imaging.

This non-contact system provides a self-calibration feature allowing measurement simplicity and stability; low-cost LED light source with no reliance on use of laser for highly coherent light; 4D big data and machine learning based image analysis; tissue optics model based blood oxygenation assay and a compact system design.

Some embodiments of the present inventive concept provide methods, systems and computer program products for non-contact four-dimensional (4D) detection of blood vessel structures and modulations of turbid media. Conventional photoplethysmography acquires scattered light signals from human tissues as a function of time to assess the blood volume changes in the microvascular bed of tissue due to the artery pulsation. Quantitative measurement and analysis of blood distribution in human tissues including skin is a very challenging problem due to the strong turbid of tissue and highly heterogeneous nature of blood capillary vessel networks mixed with other tissue chromophores. Compared to other body signals, such as electric, thermal and fluorescence, the scattered light signals are strong and relatively easy to measure. The principle of probing physiology conditions based on scattered light measurement has led to development of various widely used medical devices, such as pulse oximeter and blood pressure monitors, which have been widely used in clinics and operation rooms. While these devices are simple to make and use, they have disadvantages of limited information content, inability to determine blood oxygen distribution, and changes in blood volume and oxygenation conditions in tissues.

Significant improvement of existing optical technology for measurement of blood volume change and capillary vessel movement generally requires the ability to quantify light absorption and scattering processes, which is fundamental to understanding the complex relation between the scattered light distribution and tissue perfusion modulated by heart pulsation. Consequently, it is critically important to perform measurements in multiple domains in the form of “big data” and develop powerful tools to analyze the acquired data for extraction of accurate physiological information for clinical applications.

It has been shown that the selected absorption and scattering properties of different skin tissue components, such as melanin pigments in the visible and near infrared regions can be used for diagnosis of melanoma and other cancers. By combining reflectance imaging with spectral scans, the spatial distribution of the tissue components of interest like red blood cells moving in the capillary vessels of blood in the skin dermis layer can be determined as 3D data cube of 2D in real space and 1D in light wavelength. As discussed above, some embodiments of the present inventive concept provide a significant improvement by adding the time-domain measurement of the reflectance image data acquisition and analysis to perform 4D measurement of the tissue blood distribution and movement that allows quantitative and non-contact determination of distribution on blood pulsation and blood oxygenation. Embodiments of the present inventive concept are designed to take the advantage of “big data” nature of the 4D images to quantitatively analyze, learn and extract the blood perfusion information for clinical applications.

Some embodiments of the present inventive concept include the following advantages over the conventional technology: (1) apply derivative measurement to determine reflectance without use of reflectance standard with dIR(x,y; t;λ)/dI0=R(x,y; t;λ); (2) perform time-domain measurement of reflectance imaging as R(x,y; t; λ); (3) perform multispectral measurement of time-domain reflectance imaging as R(x,y; t; λ); (4) transform acquired data into frequency domain as R(x,y; f; λ) by Fourier transform and frequency map f(x, y; λ); (5) extract the Fourier component image of R(x,y; fh; λ) with fh=heartbeat frequency and heart-beat fh(x,y; λ); (6) perform demodulation on R(x,y; f; λ) at the frequency map fh(x,y; λ) to obtain blood volume map Vh(x,y; λ); and (7) determine blood oxygenation map Vh(x,y; λ) from its wavelength λ dependence based on radiative transfer model of tissue optics. See also, Peng Tian et al., Quantitative characterization of turbidity by radiative transfer based reflectance imaging, Biomedical Optics Express 2081, Vol. 9, No. 5, 1 May 2018, the content of which is hereby incorporated by reference as if recited in full herein.

Some embodiments of the present inventive concept have the following advantages over the conventional technology: (1) the device is of non-contact nature with the imaged tissues; (2) the device does not require any coherent light source for excitation and can be implemented with a non-coherent light source, such as LED; (3) The spectral measurement can be implemented with low-cost wavelength filters for up to about 30 wavelengths or general-use CCD or CMOS camera for 3 to 4 wavelengths with no filters; and (4) the device generally does not require a calibrated reflectance standard for tissue reflectance measurement and the measured 4D data can be compared to rigorous tissue optics model to determine inherent optical parameters of tissues and their spatial distribution, which allows highly accurate and reliable measurement of heart-beat, tissue blood perfusion and oxygenation.

Example embodiments are described above with reference to block diagrams and/or flowchart illustrations of methods, devices, systems and/or computer program products. It is understood that a block of the block diagrams and/or flowchart illustrations, and combinations of blocks in the block diagrams and/or flowchart illustrations, can be implemented by computer program instructions. These computer program instructions may be provided to a processor of a general purpose computer, special purpose computer, and/or other programmable data processing apparatus to produce a machine, such that the instructions, which execute via the processor of the computer and/or other programmable data processing apparatus, create means (functionality) and/or structure for implementing the functions/acts specified in the block diagrams and/or flowchart block or blocks.

These computer program instructions may also be stored in a computer-readable memory that can direct a computer or other programmable data processing apparatus to function in a particular manner, such that the instructions stored in the computer-readable memory produce an article of manufacture including instructions which implement the functions/acts specified in the block diagrams and/or flowchart block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the block diagrams and/or flowchart block or blocks.

Accordingly, example embodiments may be implemented in hardware and/or in software (including firmware, resident software, micro-code, etc.). Furthermore, example embodiments may take the form of a computer program product on a computer-usable or computer-readable storage medium having computer-usable or computer-readable program code embodied in the medium for use by or in connection with an instruction execution system. In the context of this document, a computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific examples (a non-exhaustive list) of the computer-readable medium would include the following: an electrical connection having one or more wires, a portable computer diskette, a random access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Computer program code for carrying out operations of data processing systems discussed herein may be written in a high-level programming language, such as Java, AJAX (Asynchronous JavaScript), C, and/or C++, for development convenience. In addition, computer program code for carrying out operations of example embodiments may also be written in other programming languages, such as, but not limited to, interpreted languages. Some modules or routines may be written in assembly language or even micro-code to enhance performance and/or memory usage. However, embodiments are not limited to a particular programming language. It will be further appreciated that the functionality of any or all of the program modules may also be implemented using discrete hardware components, one or more application specific integrated circuits (ASICs), or a field programmable gate array (FPGA), or a programmed digital signal processor, a programmed logic controller (PLC), microcontroller or graphics processing unit.

It should also be noted that in some alternate implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved. Moreover, the functionality of a given block of the flowcharts and/or block diagrams may be separated into multiple blocks and/or the functionality of two or more blocks of the flowcharts and/or block diagrams may be at least partially integrated.

In the drawings and specification, there have been disclosed example embodiments of the inventive concept. Although specific terms are employed, they are used in a generic and descriptive sense only and not for purposes of limitation, the scope of the inventive concept being defined by the following claims.

What is claimed is:

1. A system for non-contact imaging measurement of blood oxygen saturation and perfusion in a sample, the system comprising:
a control unit configured to facilitate acquisition of data from a sample;
a data acquisition module coupled to the control unit, the data acquisition module configured to illuminate a field of view (FOV) of the sample using a plurality of wavelengths to provide a plurality of images corresponding to each of the plurality of wavelengths responsive to control signals from the control unit; and
an image processing module configured calculate image saturation parameters and reflectance for each of the plurality of images having a unique acquisition time and unique wavelength and extracting blood volume and oxygen saturation data in the FOV using the calculated image saturation parameters and reflectance for each of the plurality of images having a unique acquisition time and unique wavelength.

2. The system of claim 1, wherein the data acquisition module comprises:
a plurality of sets of light emitting diodes (LEDs) each having an associated wavelength; and
a camera coupled to the plurality of sets of LEDs, wherein each set of LEDs is configured to illuminate the FOV of the sample at the associated wavelength responsive to a unique driving current from the control unit to provide an image of the FOV of the sample at the associated wavelength.

3. The system of claim 2, wherein each of the plurality of images are acquired at the associated plurality of wavelengths using a narrow bandwidth in a range from about 0.2 nm to about 50 nm.

4. The system of claim 2, wherein the camera comprises a charge coupled device (CCD) camera and wherein each of LEDs have an optical power of at least 500 mW per wavelength.

5. The system of claim 1, wherein extracting blood volume and oxygen saturation data comprises extracting heart-rate based mapping of blood vessel volume changes and detecting blood oxygen saturation level.

6. The system of claim 1 further configured to obtain a fused image of blood perfusion and oxygen saturation in skin tissues in a visible region and probe deeper tissue layers of lower dermis and cutaneous fat layers in near-infrared (NIR) regions using the plurality of images obtained at the corresponding plurality of wavelengths.

7. The system of claim 1, wherein the system is handheld.

8. The system of claim 1, wherein the system is configured to self-calibrate.

9. A non-contact method for imaging measurement of blood oxygen saturation and perfusion in a sample, the method comprising:
illuminating a field of view (FOV) of the sample using a plurality of wavelengths to provide a plurality of images corresponding to each of the plurality of wavelengths responsive to control signals from a control unit; and
calculating image saturation parameters and reflectance for each of the plurality of images having a unique acquisition time and unique wavelength; and
extracting blood volume and oxygen saturation data in the FOV using the calculated image saturation parameters and reflectance for each of the plurality of images having a unique acquisition time and unique wavelength.

10. The method of claim 9:
wherein illuminating further comprises illuminating the FOV of the sample using a plurality of sets of light emitting diodes (LEDs) each having an associated wavelength; and
wherein each set of LEDs is configured to illuminate the FOV of the sample at the associated wavelength responsive to a unique driving current from the control unit to provide an image of the FOV of the sample at the associated wavelength.

11. The method of claim 10, further comprising acquiring each of the plurality of images at the associated plurality of wavelengths using a narrow bandwidth in a range from about 0.2 nm to about 50 nm.

12. The method of claim 10, wherein the LEDs are associated with a camera, the camera comprising a charge coupled device (CCD) camera and wherein each of LEDs have an optical power of at least 500 mW per wavelength.

13. The method of claim 9, wherein extracting blood volume and oxygen saturation data comprises extracting heart-rate based mapping of blood vessel volume changes and detecting blood oxygen saturation level.

14. The method of claim 9, further comprising obtaining a fused image of blood perfusion and oxygen saturation in skin tissues in a visible region and probe deeper tissue layers of lower dermis and cutaneous fat layers in near-infrared (NIR) regions using the plurality of images obtained at the corresponding plurality of wavelengths.

15. The method of claim 9, further comprising self-calibrating a system associated with the method.

16. A computer program product for non-contact method for imaging measurement of blood oxygen saturation and perfusion in a sample, the computer program product comprising:
a non-transitory computer-readable storage medium having computer-readable program code embodied in the medium, the computer-readable program code comprising:
computer readable program code to illuminate illuminating a field of view (FOV) of the sample using a plurality of wavelengths to provide a plurality of images corresponding to each of the plurality of wavelengths responsive to control signals from a control unit; and
computer readable program code to calculate image saturation parameters and reflectance for each of the plurality of images having a unique acquisition time and unique wavelength; and
computer readable program code to extract blood volume and oxygen saturation data in the FOV using the calculated image saturation parameters and reflectance for each of the plurality of images having a unique acquisition time and unique wavelength.

17. The computer program product of claim 16:
wherein the computer readable program code to illuminate further comprises computer readable program code to illuminate the FOV of the sample using a plurality of sets of light emitting diodes (LEDs) each having an associated wavelength responsive to a unique driving current from the control unit to provide an image of the FOV of the sample at the associated wavelength.

18. The computer program product of claim 17, further comprising computer readable program code to acquire each of the plurality of images at the associated plurality of wavelengths using a narrow bandwidth in a range from about 0.2 nm to about 50 nm.

19. The computer program product of claim 16, wherein the computer readable program code to extract blood volume and oxygen saturation data comprises computer readable program code to extract heart-rate based mapping of blood vessel volume changes and detecting blood oxygen saturation level.

20. The computer program product of claim 16, further comprising computer readable program code to obtain a fused image of blood perfusion and oxygen saturation in skin tissues in a visible region and probe deeper tissue layers of lower dermis and cutaneous fat layers in near-infrared (NIR) regions using the plurality of images obtained at the corresponding plurality of wavelengths.
